(19) United States
(12) Patent Application Publication
(10) Pub. No.: US 2005/0255062 A1
(43) Pub. Date: Nov. 17, 2005

(54) Fluid substance and system and method for applying a fluid substance by airbrush

(76) Inventors: Sheila McKenna, Franklin Square, NY (US); Doug McAward, Brookfield, CT (US)

Correspondence Address: Greenberg Traurig, LLP, 22nd Floor, 885 Third Avenue, New York, NY, 10022, US

(21) Appl. No.: 10/844,657

(22) Filed: May 12, 2004

(51) Int. Cl. A61K 7/021

(52) U.S. Cl. 424/63; 118/300

(57) ABSTRACT

This invention provides a system and method a fluid substance such as makeup and a system and method for preparing the fluid substance. The invention also deals with a system and method of applying a fluid substance to a surface with an airbrush by adjusting the airflow to an airbrush by altering the power supplied to the air source.

### FIELD OF THE INVENTION

The invention concerns a fluid substance and a method for preparing the fluid substance. The invention also deals with a system and method of applying a fluid substance.

### BACKGROUND OF THE INVENTION

It is well-known to apply makeup to photography subjects and especially those subjects who will be viewed on television. Traditional makeup application, prior to the use of airbrushes for applying makeup, involved applying liquid, cream or powder makeup to the skin using a brush, fingers, or a sponge by swiping or stippling processes. Although swiping or stippling marks left on the skin by the makeup application generally are not seen on conventional television, such marks are visible on High Definition Television (HDTV).

An airbrush can be used to provide a more efficient and even makeup application. Makeup is loaded into the airbrush and then blown through a nozzle of the airbrush together with air. The makeup is deposited on the skin in tiny dots while avoiding swiping or stippling marks on the skin. However, traditional liquid makeup preparations suffer from the a number of disadvantages, including the fact that they are too thick and tend to dry out in and clog the airbrush nozzle. Even known liquid makeup preparations specifically developed for use in airbrushes suffer from the disadvantage that the pigments in the makeup tend to separate so that the preparations need to be well-shaken before introduction into the airbrush. Beads typically are provided in the makeup container to facilitate mixing of the pigments in the composition. An makeup composition, specifically for use in an airbrush, is needed which addresses the disadvantages of known makeup preparations of similar utility.

Airbrushing cosmetics has been plagued by problems which have hindered its acceptance in the cosmetics industry. Among the complaints by makeup professionals is that the available airbrush compressors are too heavy and too noisy. Another problem is “surges” resulting from incompatibility between the air source, the brush, and the product that is applied. Yet another problem is airbrush design that has external parts which are breakable. Yet another problematic issue with the current state of the art is that current air compressors either do not have adjustable airflow, or those that do are heavy, unwieldy, and require external components to adjust the air flow. These external components are subject to breakage when transported.

Adjustable airflow is important in when applying substances such as cosmetics to the skin. Facial skin requires for delicate work a soft airflow, whereas body skin requires a strong airflow.

What is needed is a device for airbrushing which is lightweight, portable, quiet, and is compact enough to fit comfortably in a small bag or case, such as a makeup kit. What is also needed is a device for airbrushing which has an adjustable airflow and yet does not require fragile external components. Yet another need is for an source of air for an airbrush which can run indefinitely without overheating and which does not require the use of an air storage tank.

### BRIEF SUMMARY OF THE INVENTION

In one embodiment the present invention is directed toward a method of applying a fluid substance to a surface. The method comprises:
(a) admixing a liquid preparation comprising water, propylene glycol, magnesium aluminum silicate, triethanolamine, cellulose gum, diazolidinyl urea, methylparaben, with a dilution amount of water to obtain the fluid substance;
(b) introducing the fluid substance thus produced into an airbrush; and
(c) applying the fluid substance to a surface with said airbrush.

This preparation and said dilution amount of water can be present in said fluid substance in amounts effective to suspend at least one pigment in said fluid substance without frequent mixing, pass through an airbrush without clogging said nozzle, and produce a substantially uniform appearance of application on the surface. Using this method, the airbrush can be operated at from about 2 psi to about 20 psi. The airbrush employed may be an Iwata HP, an Iwata Eclipse, a Badger, or a Paasche.

The fluid substance used in the method may be comprised of water, propylene glycol, magnesium aluminum silicate, triethanolanine, cellulose gum, diazolidinyl urea, methylparaben, and propylparaben. The substance may further comprise at least one of titanium dioxide, ultramarine blue, chromium oxide green, ultramarine pink, FD&C yellow #5, FD&C red #6, barium lake, FD&C red #7, calcium lake, iron oxides or talc.

In this method, the preparation and the dilution amount of water may be present in a range of from about 5:1 to about 2.5:1—for example at about 3:1. The preparation No. 111-4 (Mehron Co.) may be used.

The fluid substance of the method may further comprise glycerin. The liquid preparation, the water and the glycerin can be present in a range of from about 16:1:1 to about 4:1:1—for example about 8:1:1. The liquid preparation of the fluid substance with glycerin comprises water, propylene glycol, magnesium aluminum silicate, triethanolanine, cellulose gum, diazolidinyl urea, methylparaben, and propylparaben. The liquid preparation may yet further comprise at least one of titanium dioxide, ultramarine blue, chromium oxide green, ultramarine pink, FD&C yellow #5, FD&C red #6, barium lake, FD&C red #7, calcium lake, iron oxides or talc. No. 111-4 (Mehron Co.) may be used as the liquid preparation. The airbrush employed may be an Iwata HP, an Iwata Eclipse, a Badger or a Paasche. The airbrush can be operated at from about 2 psi to about 20 psi—for example at about 6 psi.

The fluid substance is adapted to allow the airbrush to be effectively cleaned using only water after the fluid substance has passed through the airbrush. Using this fluid substance, the substantially uniform appearance of application on the surface is substantially uniform as viewed on high definition television.

In another embodiment is presented a method of making a fluid substance for application to a surface comprising admixing a liquid preparation and a dilution amount of water in amounts effective to enable the fluid substance to pass through an airbrush without clogging the airbrush, wherein said fluid substance is adapted to produce a substantially uniform appearance of application on the surface. In the method the liquid preparation and the dilution amount of water can be employed in amounts effective to suspend at least one pigment in the fluid substance. The fluid substance may be a cosmetic which may further be substantially odorless.

The liquid preparation employed by the method may comprise water, propylene glycol, magnesium aluminum silicate, triethanolanine, cellulose gum, diazolidinyl urea, methylparaben, and propylparaben. It may further comprise at least one of titanium dioxide, ultramarine blue, chromium oxide green, ultramarine pink, FD&C yellow #5, FD&C red #6, barium lake, FD&C red #7, calcium lake, iron oxides or talc. According to the method, the liquid preparation and the dilution amount of water can be present in a range of from about 5:1 to about 2.5:1—about 3:1 for example. The liquid preparation may be No. 111-4 (Mehron Co.).

According to the method, the fluid substance may comprise glycerin. When so comprised, the liquid preparation and the dilution amount of water can be employed in amounts effective to suspend at least one pigment in the fluid substance. The fluid substance may be a movable cosmetic. The liquid preparation, the dilution amount of water and the glycerin can be present in a range of from about 16:1:1 to about 4:1:1, about 8:1:1 for example. The liquid preparation employed by the method may comprise water, propylene glycol, magnesium aluminum silicate, triethanolanine, cellulose gum, diazolidinyl urea, methylparaben, and propylparaben. It may further comprise at least one of titanium dioxide, ultramarine blue, chromium oxide green, ultramarine pink, FD&C yellow #5, FD&C red #6, barium lake, FD&C red #7, calcium lake, iron oxides or talc. According to the method, the liquid preparation and the dilution amount of water can be present in a range of from about 5:1 to about 2.5:1—about 3:1 for example. The liquid preparation may be No. 111-4 (Mehron Co.).

This fluid substance can be adapted to allow the airbrush to be effectively cleaned using only water after the fluid substance has passed through the airbrush. The substantially uniform appearance application on the surface is substantially uniform as viewed on high definition television.

In another embodiment of the present invention, a fluid substance for application to a surface is provided. The fluid substance can comprise: a liquid preparation and a dilution amount of water, wherein the liquid preparation and dilution amount of the water are present in amounts effective to pass through an airbrush without clogging the airbrush; and produce a substantially uniform appearance of application when applied to a surface.

The liquid preparation and the dilution amount of water can be present in amounts effective to suspend to at least one pigment in the fluid substance without frequent mixing. The fluid substance can be adapted to pass through the airbrush without clogging the airbrush when the flow of air through the airbrush is from about 2 psi to about 20 psi. The fluid substance may be a cosmetic. It can be substantially odorless. It may further comprise glycerin and may be a movable cosmetic. The fluid substance may be non transferable. It can also be adapted to allow the airbrush to be effectively cleaned using only water after the fluid substance has passed through the airbrush. The dilution amount of water may be filtered water.

The fluid substance's liquid preparation can comprise water; propylene glycol, magnesium aluminum silicate, triethanolanine, cellulose gum, diazolidinyl urea, methylparaben, and propylparaben. The liquid preparation may further comprise at least one of titanium dioxide, ultramarine blue, chromium oxide green, ultramarine pink, FD& C yellow #5, FD&C red #6, barium lake, FD&C red #7, calcium lake, iron oxide or talc. The liquid preparation and the dilution amount of water of the fluid substance may be present in a range of from about 5:1 to about 2.5:1—for example about 3:1. The liquid preparation may be No. 111-4 (Mehron Co.).

Where the fluid substance comprises glycerin, the liquid preparation, the dilution amount of water and the glycerin can be present in the range of from about 16:1:1 to about 4:1:1-8:1:1 for example. The fluid substance comprising glycerin can have a liquid preparation comprising water, propylene glycol, magnesium aluminum silicate, triethanolanine, cellulose gum, diazolidinyl urea, methylparaben, and propylparaben. The liquid preparation may further comprise at least one of titanium dioxide, ultramarine blue, chromium oxide green, ultramarine pink, FD& C yellow #5, FD&C red #6, barium lake, FD&C red #7, calcium lake, iron oxide or talc. It may be No. 111-4 (Mehron Co.).

In yet another embodiment is presented a fluid substance adapted to pass through a nozzle of a conventional airbrush to appear substantially uniform on a surface, said fluid substance comprising a dilution liquid in an amount of between about 5:1 to about 2.5:1 with respect to said fluid substance. The dilution liquid may comprise water. The fluid substance may further comprise glycerin. The fluid substance can be adapted to allow the airbrush to be effectively cleaned using only water after the fluid substance has passed through the airbrush. The substantially uniform appearance on the surface can be substantially uniform as viewed on high definition television.

The present invention provides a device for applying a fluid substance to a surface comprising:
(a) an air source
(b) an airbrush operatively connected to said air source by a hose;
(c) a controller operatively connected to said air source for adjusting power to the air source; wherein a flow of air from the air source is adjusted by adjusting the power supplied to the air source.

The air source may be adapted to be powered at from about 3 volts to about 14 volts. The air source may be adapted to provide the flow of air to the airbrush at from about 3 psi to about 14 psi, wherein the volt to psi ratio is about 1:1. Where the air source is adapted to provide the flow of air at from about 3 psi to about 6 psi and the voltage is from about 3 volts to about 6 volts, the device may be adapted to be used to apply the fluid substance to a subject's face. Where the air source is adapted to provide the flow of air at from about 7 psi to about 14 psi and the voltage is from about 7 volts to about 14 volts, the device can be adapted to be used to apply the fluid substance to a subject's body. The air source need not overheat when supplied with power. The air source may be no louder than about 68 to 79 dB when provided with power.

One aspect of the device is that it is lightweight, portable and substantially smooth insofar as there are no fragile external parts (e.g.: an airflow regulator). It's construction may be such that it is can fit comfortably within small case, such as a makeup professional's makeup bag, and can be carried without strain or effort. For example, the device can further comprise a housing, and it may be no more than about 2.5 pounds. The housing for the air source may be substantially cuboid, having a volume of no more than about 8″ by 3 1/2″ by 3 1/2″. The housing can be substantially smooth and may include an opening for holding the airbrush.

The device may be used to apply the fluid substance to a subject's skin. The fluid substance may be water-based to prevent clogging of the airbrush. This fluid substance can also be a cosmetic. It may further comprise glycerin, and it may be a movable cosmetic.

In another embodiment, a method of controlling airflow to an airbrush comprising adjusting the power supplied to an air source is provided. The method further comprises powering the air source at from about 3 volts to about 14 volts. The air source may provide the airflow at from about 3 psi to about 14 psi, the psi and the voltage powering the air source being at about a 1 to 1 ratio. Where the voltage is from about 3 volts to about 6 volts and the airflow is from about 3 psi to about 6 psi, the airbrush may be used to apply a fluid substance to a subject's face. Where the voltage is from about 7 volts to about 14 volts and the airflow is from about 7 psi to about 14 psi., the airbrush may be used to apply the fluid substance to a subject's body.

### BRIEF DESCRIPTION OF THE DRAWING

FIG. 1 is a perspective view, partially cut away, of the present invention.

### DETAILED DESCRIPTION OF THE INVENTION

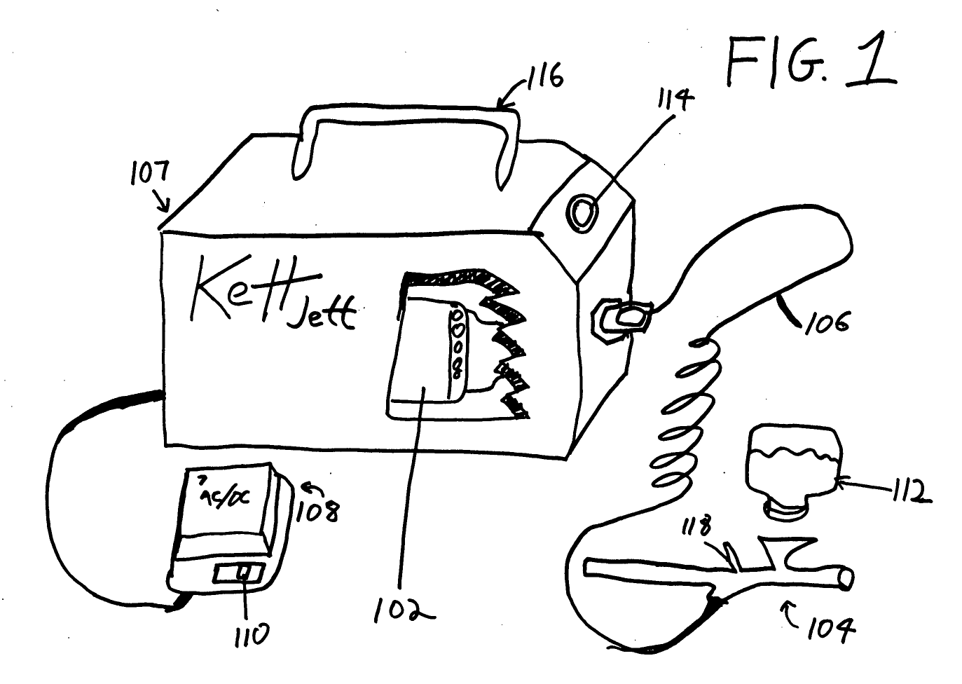

FIG. 1 shows one embodiment of an invention for applying a fluid substance to a surface, such as makeup to a person's face or body for example.

An airbrush 104 is connected to an air source 102 by a hose. The air source 102 may be secured in a housing 107. Power is conducted to the air source 102 through a controller 108 which can alter the power supplied to the air source 102.

The housing 107 is made with a 1 and 5/8 inch opening 114 with a grommet adapted to hold the airbrush upright. In this embodiment the air source 102 is an Oken Seiko Micro Air Pump # P30-C-0002 (with capacitors). The air source 102, when powered, provides via a hose 106 an airflow to an airbrush. This air source is no louder than about 68 to 79 dB in the absence of any muffling, and when powered using lower voltages is significantly quieter. The Iwata HP B airbrush is an example an airbrush useful for applying a fluid substance such as a cosmetic, as it has the one of finest nozzles available and is especially useful for fine detail with a delicate spray pattern. An Iwata Eclipse, Badger or Paasche could also be used.

The air source 102 may be inside a portable, cuboid housing 107. The power to the air source 102 can be altered by a controller 108. The controller 108 can be an Adjustable Universal Switchable Power Supply (3 to 14 volt output/110 to 240 volt input). The controller 108 allows power to be provided to the air source 102 at anywhere from 3 volts to 14 volts by virtue of a volt change switch 110.

The air source 102 is capable of operating when provided with power at variable voltages, as is described below. The micro air pump of the present embodiment does not require the use of air storage tank or an external device to regulate the flow of air. When provided with power at a constant voltage, the pump produces a steady air-flow to the airbrush. By employing a controller 108, it was determined that the air flow of an air source 102 can be controlled by altering the power, in this case voltage, provided to the air source 102. Using a psi gauge, it was determined that the micro pump generates air flow psi at a 1 to 1 ratio with voltage provided it. Thus when 3 volts are supplied to the micro air pump, the micro air pump in turn produces an air flow of about 3 psi. In the embodiments 4.5 volts generates about 4.5 psi, 9 volts generates about 9 psi, and so on. This is especially useful when applying a fluid substance to a subject's skin.

A fluid substance such as a cosmetic is optimally applied to a subject's skin using different airflows when applied by an airbrush. What airflow is used depends on where on the subject's skin the fluid substance is to be applied. For example, a cosmetic such as makeup is optimally applied to the person's face using a lower flow of air, such as from about 3 psi to about 6 psi. Yet if the cosmetic is to be applied to the body, then the optimal air flow is stronger, for example from about 7 psi to about 14 psi. The invention makes it possible to create these varying air flows by varying the power supplied to the air source 102. In this embodiment the air flow is produced at an about 1:1 ratio with voltage.

The invention may include a housing 107 to enclose the air source 102. The housing can be substantially cuboid in shape, having overall dimensions of about 8″ in width by 3 and ½″ and 3 and ½″ in height (not including the handle 116). The housing 107 can be designed to be substantially smooth such that there are no external components subject to breakage. A durable handle 116 may be included for added portability. The housing 107 can also include an opening 114 to hold the airbrush 104 when not in use. A 1 and ⅝″ opening 114 is shown. The opening 114 includes a grommet for holding the airbrush upright. Another opening allows air into the housing for the proper operation of the air source. The housing 107 can be constructed with ABA R59 UL 94V0 suede at a thickness of 0.125″ to achieve the desired low weight. The housing 107 along with 9 rubber spacers (not shown) which both hold the air source 102 in place act as baffles which muffle the air source when in operation. Four rubber feet (not shown) which are attached to the base of the device also contribute to quieter operation by softening the vibratory effect when the device placed on a surface (e.g.: a tabletop)

In the embodiment shown, the invention is lightweight. It may be no more than abut 2.5 pounds, which aids in portability. It also is relatively quiet as compared to other airbrush devices. When in operation the air source may be no louder than about 68 to 79 dB without muffling. Because the device can provide air flow at lower voltages—about 14 volts for example—it can be powered indefinitely without overheating the air source.

In another embodiment of the invention a method of controlling airflow to an airbrush is provided. The method comprises altering the power supplied to an air source such that the flow or air is altered when the power is altered. As shown in the present invention, by an air source is provided with anywhere from about 3 to about 14 volts. This results in the air source providing an airflow from about 3 to about 14 psi at a 1:1 ratio with the voltage provided.

Where the method is used to provide an airflow of anywhere within the range of from about 3 psi to about 6 psi by altering the power of anywhere within the range of from about 3 volts to about 6 volts, the airbrush may be used to apply a fluid substance to a subject's face. Where the method is used to provide an airflow of anywhere within the range of from about 7 psi to about 14 psi by altering the power of anywhere within the range of from about 7 volts to about 14 volts, the method may be used to apply a fluid substance of a subject's body.

The invention can be used to apply any fluid substance 112 to any surface, such as when it is used to apply a cosmetic to a subject's skin. The invention described can be used to apply a movable cosmetic. “Movable” as applied to cosmetics, means a cosmetic that is, among other things, blendable. A cosmetic that is not movable is unforgiving, since when it is applied to a subject's skin it cannot be blended or adjusted. This means that one applying the cosmetic cannot make an error, since the only means of correction is to remove all of the cosmetic and begin anew. A movable cosmetic, on the other hand, allows an individual applying the cosmetic to make adjustments while in the process of application. This is vitally important aspect of applying cosmetics such as make-up, since achieving the desired result is often accomplished by virtue of adjustments made in the process of application.

The invention provides a fluid substance for application to a subject's skin which is capable of smoothly passing through an airbrush without clogging it. The fluid substance may be a cosmetic. The cosmetic may be movable, and it may also be substantially odorless.

According to a first aspect of the invention, the fluid substance for use in an airbrush comprises a liquid preparation to which has been added a dilution amount of a liquid such as water. The fluid substance can be a cosmetic. The cosmetic may be of the type comprising water, propylene glycol, magnesium aluminum silicate, triethanolanine, cellulose gum, diazolidinyl urea, methylparaben, and propylparaben. The fluid substance can be water based, not water polymer based.

The preparation and the dilution amount of liquid are present in amounts effective to maintain pigments suspended in the composition without frequent mixing, to pass through an airbrush nozzle without clogging, and to produce a substantially uniform appearance of application on the skin when the makeup composition is applied to skin by means of an airbrush.

The preparation may further include any of titanium dioxide, ultramarine blue, chromium oxide green, ultramarine pink, FD&C yellow #5, FD&C red #6, barium lake, FD&C red #7, calcium lake, iron oxides and talc.

In one embodiment of the invention, the liquid preparation employed in the invention is number 111-4 (Mehron Co., Chestnut Ridge, N.Y.).

Generally, the ratio of the makeup preparation to the dilution amount of liquid in the composition is in the range of about 5:1 to about 2.5:1. The range can be about 3:1, for example.

In another embodiment of the first aspect of the invention, glycerin is added to the fluid substance in amounts effective to modify the consistency of the fluid substance where the fluid substance is a cosmetic, this enables easier spot correction on a subject's skin once the cosmetic has been applied, and/or to provide the subject with a dewier appearance.

Glycerin is especially useful in making a cosmetic “moveable”. Silicone is a very popular product to use in cosmetic makeup. Silicone fills gaps when a cosmetic is applied to the skin, such as facial skin. However, once the skin moves, such as when a person smiles, the silicone moves out of the cracks and leaves a noticeable marring mark, such as streak of makeup on the face. Nor can the mark be powdered over, since silicone will not hold the powder. Glycerin, on the other hand, is water-soluble. As such, it can “grab” the powder and the powder will stay where it is applied.

Generally, the ratio of the preparation, the dilution liquid and the glycerin in the composition will be in the range of about 16:1:1 to about 4:1:1. Preferably, the range is about 8:1:1.

According to a second aspect of the invention, a method of making the fluid substance, in one embodiment, admixing a liquid preparation of the type comprising water, propylene glycol, magnesium aluminum silicate, triethanolamine, cellulose gum, diazolidinyl urea, methylparaben, and propylparaben, with a dilution amount of liquid such as water in amounts sufficient, and for a time sufficient, to maintain the pigments suspended in the composition without frequent mixing, to pass through an airbrush without clogging, and to produce a uniform appearance of application on the skin when said fluid substance is applied to the skin by means of an airbrush.

In further embodiment of the second aspect of the invention, glycerin is added to the fluid substance in the amounts discussed above.

According to a third aspect of the invention, a method for applying the aforementioned fluid substance to the skin comprises:
(a) admixing a liquid preparation of the type comprising water, propylene glycol, magnesium aluminum silicate, triethanolamine, cellulose gum, diazolidinyl urea, methylparaben, and propylparaben, with a dilution amount of water to obtain a fluid substance
(b) introducing the fluid substance thus produced into an airbrush;
(c) applying said fluid substance to skin with said airbrush in such manner as to produce a uniform appearance of application on the skin.

The fluid substance also may include any of titanium dioxide, ultramarine blue, chromium oxide green, ultramarine pink, FD&C red #7, calcium lake, iron oxides and talc.

In one embodiment of the invention, the liquid preparation employed is number 111-4 (Mehron Co., Chestnut Ridge, N.Y.).

The fluid substance of the present invention can be employed in any airbrush, or even manually. Another feature is that the airbrush making use of the fluid substance can be cleaned using only water. A specialized airbrush-cleaning product is not required. Airbrush devices which may be employed in said method include, but are not limited to: an Iwata HP, an Iwata Eclipse, a Badger, or a Paasche.

### EXAMPLE

In this experiment, a comparative liquid preparation, makeup (comparative preparation number 111-4 (Mehron Co., Chestnut Ridge, N.Y.), and the fluid substance according to the invention comprising the number 111-4 preparation, water and glycerin in a ratio of 4:1:1 were tested for flow through an Iwata HPB airbrush with an Iwata/Medea 20000 compressor at 2-20 psi, and for uniformity of application on a glass test surface. Results are shown in Tables 1 and 2.

TABLE 1
Comparative Liquid Preparation

| Psi | results |
|---|---|
| 2 | Fluid is released sporadically from the |
| | airbrush in splotches and will only release |
| | when the main lever is pulled backward and |
| | forward constantly. Otherwise the fluid |
| | ceases to be release. |
| 4 | Same as 2 psi. |
| 6 | Same as 2 psi. |
| 8 | Same as 2 psi. |
| 10 | Fluid sprays evenly from the airbrush only |
| | when main lever is pulled back to full force. |
| | When the main lever is slightly released, a |
| | splotch of liquid is released resulting in an |
| | uneven makeup application that can only be |
| | applied at 10 psi full force. |
| 12 | Same as 10 psi. |
| 14 | Fluid is released from the airbrush when |
| | lever is pulled back ¾ths of the way. |
| | Splotches appear when the lever is release. |
| 16 | Fluid is released when lever is pulled back |
| | ½ the way. Splotches appear when the lever |
| | is released. |
| 18 | Fluid is released when the lever is pulled |
| | back ⅜ths of the way. Splotches appear |
| | when the lever is released. |
| 20 | Same as 18 psi. |

The above data demonstrate that the results obtained are poor. The cosmetic needs to flow evenly from the airbrush when the main lever is pulled back and forth. The cosmetic needs to be released from the airbrush at a constant rate, but here it is different at each psi setting and at each position of the lever. When Mehron Co. liquid makeup was sprayed onto a glass test surface at 6 psi, under 96×144× magnification, the pixels were uneven. There was no uniformity to the application.

TABLE 2
Inventive Fluid Substance

| Psi | Result |
|---|---|
| 2 | No splatter, no splotch, even flow at all |
| | positions with main lever. |
| 4 | Same as 2 psi. |
| 6 | Same as 2 psi. |
| 8 | Same as 2 psi. |
| 10 | Same as 2 psi. |
| 12 | Same as 2 psi. |
| 14 | Same as 2 psi. |
| 16 | Same as 2 psi. |
| 18 | Same as 2 psi. |
| 20 | Same as 2 psi. |

The results of the test were excellent. This formula worked well at all positions on the compressor regulator between 2 and 20 psi and at all positions of the main lever in the airbrush, When the formula was magnified in a glass test surface at 96×144× magnification, an even layer of different sized pixels and shapes was obtained, mirroring the complexion when magnified.

Although the present invention has been described in relation to particular preferred embodiments thereof, many variations and modifications and other uses may be made without departing from the invention. Accordingly, it is intended that all such alterations and modifications be included within the spirit and scope of the invention as defined in the appended claims.

1. A cosmetic fluid substance for application to a surface comprising:
a liquid preparation and a dilution amount of water, wherein the liquid preparation and dilution amount of the water are present in amounts effective to
(a) pass through an airbrush without clogging the airbrush; and
(b) produce a substantially uniform appearance of application when applied to a surface.

2. The fluid substance of claim 1 wherein the liquid preparation and the dilution amount of water are present in amounts effective to suspend to at least one pigment in the fluid substance without frequent mixing.

3. The fluid substance of claim 1 wherein the fluid substance is adapted to pass through the airbrush without clogging the airbrush when the flow of air through the airbrush is from about 2 psi to about 20 psi.

4. The fluid substance of claim 1 wherein the fluid substance is a movable cosmetic.

5. The fluid substance of claim 4 wherein the fluid substance further comprises glycerin.

6. The fluid substance of claim 1 wherein the substance is substantially odorless.

7. The fluid substance of claim 1, wherein said liquid preparation comprises water; propylene glycol, magnesium aluminum silicate, triethanolanine, cellulose gum, diazolidinyl urea, methylparaben, and propylparaben.

8. The fluid substance according to claim 7, wherein the liquid preparation further comprises at least one of titanium dioxide, ultramarine blue, chromium oxide green, ultramarine pink, FD& C yellow #5, FD&C red #6, barium lake, FD&C red #7, calcium lake, iron oxide or talc.

9. The fluid substance according to claim 1, wherein the liquid preparation and the dilution amount of water are present in a range of from about 5:1 to about 2.5:1.

10. The fluid substance according to claim 9, wherein said range is about 3:1.

11. The fluid substance according to claim 5, wherein the liquid preparation, the dilution amount of water and the glycerin are present in the range of from about 16:1:1 to about 4:1:1.

12. The fluid substance according to claim 11, wherein the range is about 8:1:1.

13. The fluid substance according to claim 5, wherein the liquid preparation comprises water, propylene glycol, magnesium aluminum silicate, triethanolanine, cellulose gum, diazolidinyl urea, methylparaben, and propylparaben.

14. The fluid substance according to claim 13, wherein the liquid preparation further comprises at least one of titanium dioxide, ultramarine blue, chromium oxide green, ultramarine pink, FD& C yellow #5, FD&C red #6, barium lake, FD&C red #7, calcium lake, iron oxide or talc.

15. The fluid substance according to claim 1, wherein the fluid is non transferable.

16. The fluid substance according to claim 1, wherein the dilution amount of water is filtered water.

17. The fluid substance according to claim 1, wherein the fluid substance is adapted to allow the airbrush to be effectively cleaned using only water after the fluid substance has passed through the airbrush.

18. A cosmetic fluid substance adapted to pass through a nozzle of a conventional airbrush to appear substantially uniform on a surface, said fluid substance comprising a dilution liquid in an amount of between about 5:1 to about 2.5:1 with respect to said fluid substance.

19. The fluid substance according to claim 18, wherein said dilution liquid comprises water.

20. The fluid substance according to claim 18, wherein said fluid substance further comprises glycerin.

21. The fluid substance of claim 18, wherein the fluid substance is adapted to allow the airbrush to be effectively cleaned using only water after the fluid substance has passed through the airbrush.

22. The fluid substance of claim 18, wherein the substantially uniform appearance on the surface is substantially uniform as viewed on high definition television.

23. A device for applying a fluid substance to a surface comprising:
(a) an air source
(b) an airbrush operatively connected to said air source by a hose;
(c) a controller operatively connected to said air source for adjusting power to the air source;
wherein a flow of air from the air source is adjusted by adjusting the power supplied to the air source.

24. The device of claim 23 wherein the device further comprises a housing enclosing at least the air source.

25. The device of claim 23 wherein the controller is adapted to adjust power to the air source such that a volt to airflow psi ratio is about 1:1.

26. The device of claim 23 wherein the surface includes a subject's skin and the device is adapted to be used to apply the fluid substance to the skin.

27. The device of claim 26 wherein the controller is adapted to adjust power to the air source at from about 3 volts to about 14 volts such that the flow of air to the airbrush from the air source is at from about 3 psi to about 14 psi.

28. The device of claim 26 wherein the device is adapted to be used to apply the fluid substance to a subject's face.

29. The device of claim 28 wherein the flow of air is at from about 3 psi to about 6 psi and the voltage is from about 3 volts to about 6 volts.

30. The device of claim 26 wherein the device is adapted to be used to apply the fluid substance to a subject's body.

31. The device of claim 30 wherein controller is adapted to adjust power to the air source such that the flow of air is at from about 7 psi to about 14 psi and the voltage is at from about 7 volts to about 14 volts.

32. The device of claim 23 wherein the air source does not overheat when supplied with power.

33. The device of claim 24 wherein the device is adapted to fit in a portable case.

34. The device of claim 24 wherein the device is lightweight such that it can be hand-carried.

35. The device of claim 34 wherein the device is no more than about 2.5 pounds.

36. The device of claim 24 wherein the housing is substantially cuboid.

37. The device of claim 33, said device having a volume of no more than about 8″ by 3½″ by 3½″.

38. The device of claim 24 wherein the housing includes an opening for holding the airbrush.
